(19) United States
(12) Patent Application Publication
(10) Pub. No.: US 2011/0184268 A1
(43) Pub. Date: Jul. 28, 2011

(54) Method, Device and System for Providing Analyte Sensor Calibration

(75) Inventor: Marc Barry Taub, Mountain View, CA (US)

(73) Assignee: Abbott Diabetes Care Inc., Alameda, CA (US)

(21) Appl. No.: 13/011,898

(22) Filed: Jan. 22, 2011

Related U.S. Application Data
(60) Provisional application No. 61/297,603, filed on Jan. 22, 2010.
(60) Provisional application No. 61/297,612, filed on Jan. 22, 2010.

(51) Int. Cl. A61B 5/145; G01N 33/48

(52) U.S. Cl. 600/365; 73/1.02

(57) ABSTRACT

Methods and devices for providing calibration in analyte monitoring systems are provided.

### RELATED APPLICATIONS

The present application claims the benefit of U.S. Provisional Patent Application No. 61/297,603 filed Jan. 22, 2010, entitled “Enhanced Calibration Using Calibrations at Different Analyte Levels” and U.S. Provisional Patent Application No. 61/297,612 filed Jan. 22, 2010, entitled “Using Testing Site Information for Enhanced Calibration”, the disclosures of each of which are incorporated herein by reference in their entirety for all purposes.

### BACKGROUND

Monitoring of the level of glucose or other analytes, such as lactate or oxygen, in certain individuals is vitally important to their health. High or low levels of glucose or other analytes may have detrimental effects. Monitoring of glucose is particularly important to individuals with diabetes. Diabetics may need to monitor glucose levels to determine when insulin is needed to reduce glucose levels in their bodies or when additional glucose is needed to raise the level of glucose in their bodies. In non-diabetic individuals, it may be important to monitor glycemic responses to determine whether therapeutic approaches may be useful to prevent the onset of diabetes.

Analyte monitoring systems may be designed to test blood samples taken periodically and measured outside of the body (in vitro testing), such as by putting a drop of blood on a test strip, and performing an analyte analysis on the test strip. Tests performed in such a manner may be referred to as “discrete” measurements, and in the case of glucose measurements, “blood glucose” (BG) measurements. Blood may be taken from a finger (by performing a “fingerstick”) or other locations on the body, such as the arm, thigh, etc. However, a glucose level reading taken from a finger-stick may be different than one taken at the thigh. Therefore, there exists a need for an analyte monitoring device which stores not only the blood glucose level, but also the location testing site.

In some situations, it is medically desirable to monitor analyte levels in a subject closely, over a substantial period of time, or on an ongoing basis for an extended time period, in some cases indefinitely. Some systems are designed to measure analyte levels within the body (in vivo), using a suitable sensor, without drawing blood for every measurement. A monitor that tracks glucose levels by automatically taking periodic in vivo measurements, e.g., one measurement per minute, or more or less frequently, is known as a “continuous glucose monitor” (CGM). Prior art CGMs have been provided, for example, in the form of a system. A portion of the system, comprising an electrochemical sensor partially inserted into the skin, and an associated processor and transmitter, with a self-contained power supply, is attached to the body of the user and will remain in place for an extended period of hours, days, weeks, etc. The transmitter takes analyte measurements periodically and transmits them, for example, by short-range radio communications, to a separate receiver/display device. The receiver/display device will typically receive discrete BG measurements (e.g., from a separate BG meter or an included BG test strip port), as well as a port, such as a USB port, for communications with upstream computers and/or other electronics. In some embodiments, the receiver unit may be directly or indirectly interfaced with an insulin pump, for managing the subject's insulin therapy

The accuracy of the analyte measurements obtained with an in vivo monitoring system is important. Calibration of such systems may be performed by comparing in vivo “system” measurements against discrete BG “reference” measurements from fingerstick samples measured on a test strip.

For CGM systems that utilize two (or more) points for calibration, the accuracy of the calibration can be improved by maximizing the distance between the calibration points. For example, a two point calibration with points at 100 mg/dL and 120 mg/dL will be less accurate, in general, than a two point calibration with calibration points at 90 mg/dL and 140 mg/dL. Accordingly, a calibration system which maximizes the distance between calibration points is desirable. It would be further desirable to utilize naturally occurring extreme glucose values (e.g., from a hypoglycemic or hyperglycemic event) as calibration points.

### INCORPORATION BY REFERENCE

Patents, applications and/or publications described herein, including the following patents, applications and/or publications are incorporated herein by reference for all purposes: U.S. Pat. Nos. 4,545,382; 4,711,245; 5,262,035; 5,262,305; 5,264,104; 5,320,715; 5,356,786; 5,509,410; 5,543,326; 5,593,852; 5,601,435; 5,628,890; 5,820,551; 5,822,715; 5,899,855; 5,918,603; 6,071,391; 6,103,033; 6,120,676; 6,121,009; 6,134,461; 6,143,164; 6,144,837; 6,161,095; 6,175,752; 6,270,455; 6,284,478; 6,299,757; 6,338,790; 6,377,894; 6,461,496; 6,503,381; 6,514,460; 6,514,718; 6,540,891; 6,560,471; 6,579,690; 6,591,125; 6,592,745; 6,600,997; 6,605,200; 6,605,201; 6,616,819; 6,618,934; 6,650,471; 6,654,625; 6,676,816; 6,730,200; 6,736,957; 6,746,582; 6,749,740; 6,764,581; 6,773,671; 6,881,551; 6,893,545; 6,932,892; 6,932,894; 6,942,518; 7,041,468; 7,167,818; 7,299,082; and 7,866,026; U.S. Published Application Nos. 2004/0186365; 2005/0182306; 2006/0025662; 2006/0091006; 2007/0056858; 2007/0068807; 2007/0095661; 2007/0108048; 2007/0199818; 2007/0227911; 2007/0233013; 2008/0066305; 2008/0081977; 2008/0102441; 2008/0148873; 2008/0161666; 2008/0267823; 2009/0054748; 2009/0247857; 2009/0294277; 2010/0081909; 2010/0198034; 2010/0213057; 2010/0230285; 2010/0313105; 2010/0326842; and 2010/0324392; U.S. patent application Ser. Nos. 12/807,278; 12/842,013; and 12/871,901; and U.S. Provisional Application Nos. 61/238,646; 61/246,825; 61/247,516; 61/249,535; 61/317,243; 61/345,562; and 61/361,374.

### SUMMARY

An analyte monitoring device in certain embodiments include an operative component adapted to measure an analyte concentration from a sample obtained from a testing location of a user, and a receiver adapted to receive a signal from the operative component relative to the measured analyte concentration, where the receiver is configured to store information corresponding to the analyte concentration and the testing location to process analyte related signals based at least in part on the stored analyte concentration information and the testing location information.

A method for calibrating an analyte sensor, comprising retrieving a first calibration measurement, requesting a current calibration measurement, receiving the current calibration measurement, comparing the first calibration measurement to the current calibration measurement, and calibrating the analyte sensor based on one or more of the retrieved first calibration measurement or the received current calibration measurement if the current calibration measurement is outside a threshold value compared to the first calibration measurement.

These and other objects, features and advantages of the present disclosure will become more fully apparent from the following detailed description of the embodiments, the appended claims and the accompanying drawings.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 illustrates a block diagram of a data monitoring and management system in certain embodiments of the present disclosure;

FIG. 2 is a block diagram of a receiver unit in certain embodiments of the present disclosure;

FIG. 3 illustrates a touch screen interface used to select a testing site in accordance with certain embodiments of the present disclosure;

FIGS. 4 and 5 are flowcharts illustrating calibration methods in accordance with certain embodiments of the present disclosure;

FIGS. 6 and 7 are flowcharts illustrating calibration processing routines in certain embodiments of the present disclosure;

FIG. 8 is a flowchart illustrating calibration processing routines in certain embodiments in connection with calibration;

FIG. 9 is a flowchart illustrating calibrating routines in an on-demand analyte monitor; and

FIG. 10 is a flowchart illustrating calibration routines in an analyte monitoring system.

### DETAILED DESCRIPTION

Before the present disclosure is described in detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The figures shown herein are not necessarily drawn to scale, with some components and features being exaggerated for clarity.

Various exemplary embodiments of the analyte monitoring system and methods of the disclosure are described in further detail below. Although the disclosure is described primarily with respect to a glucose monitoring system, each aspect of the disclosure is not intended to be limited to the particular embodiment so described. Accordingly, it is to be understood that such description should not be construed to limit the scope of the disclosure, and it is to be understood that the analyte monitoring system can be configured to monitor a variety of analytes, as described below.

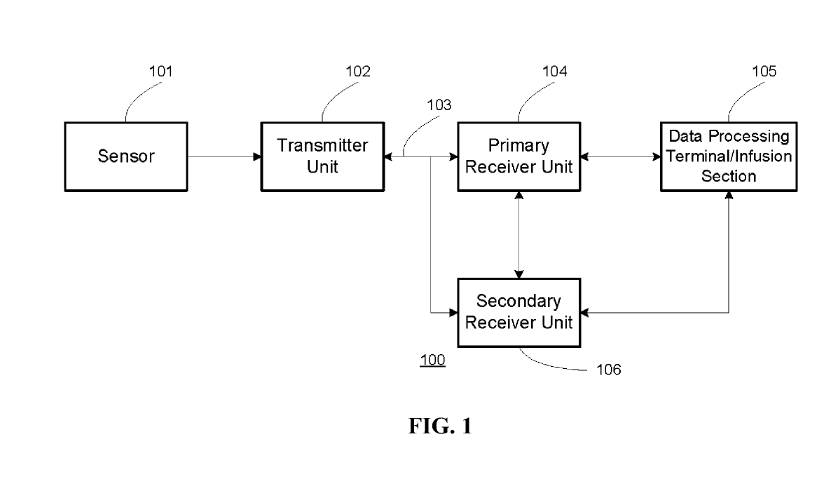

FIG. 1 illustrates a data monitoring and management system such as, for example, analyte (e.g., glucose) monitoring system 100 in accordance with embodiments of the present disclosure. In certain embodiments, the analyte monitoring system 100 may be a continuous monitoring system, a semi-continuous monitoring system, a discrete monitoring system or an on-demand monitoring system. The analyte monitoring system 100 includes a sensor 101, a transmitter unit 102 coupleable to the sensor 101, and a primary receiver unit 104 which is configured to communicate with the transmitter unit 102 via a bi-directional communication link 103. The primary receiver unit 104 may be further configured to transmit data to a data processing terminal 105 for evaluating the data received by the primary receiver unit 104. Data processing terminal 105 may include an infusion section, such that data processing terminal 105 acts as an infusion device, such as an insulin pump. Moreover, the data processing terminal 105 in one embodiment may be configured to receive data directly from the transmitter unit 102 via a communication link which may optionally be configured for bi-directional communication. Accordingly, transmitter unit 102 and/or receiver unit 104 may include a transceiver.

Also shown in FIG. 1 is an optional secondary receiver unit 106 which is operatively coupled to the communication link and configured to receive data transmitted from the transmitter unit 102. Moreover, as shown in the Figure, the secondary receiver unit 106 is configured to communicate with the primary receiver unit 104 as well as the data processing terminal 105. Indeed, the secondary receiver unit 106 may be configured for bidirectional wireless communication with each or one of the primary receiver unit 104 and the data processing terminal 105. In one embodiment of the present disclosure, the secondary receiver unit 106 may be configured to include a limited number of functions and features as compared with the primary receiver unit 104. As such, the secondary receiver unit 106 may be configured substantially in a smaller compact housing or embodied in a device such as a wrist watch, pager, mobile phone, Personal Digital Assistant (PDA), for example. Alternatively, the secondary receiver unit 106 may be configured with the same or substantially similar functionality as the primary receiver unit 104. The receiver unit may be configured to be used in conjunction with a docking cradle unit, for example for one or more of the following or other functions: placement by bedside, for re-charging, for data management, for night time monitoring, and/or bidirectional communication device.

In one aspect sensor 101 may include two or more sensors, each configured to communicate with transmitter unit 102. Furthermore, while only one, transmitter unit 102, communication link 103, and data processing terminal 105 are shown in the embodiment of the analyte monitoring system 100 illustrated in FIG. 1, in certain embodiments, the analyte monitoring system 100 may include one or more sensors, multiple transmitter units 102, communication links 103, and data processing terminals 105. Moreover, within the scope of the present disclosure, the analyte monitoring system 100 may be a continuous monitoring system, or semi-continuous, or a discrete monitoring system. In a multi-component environment, each device is configured to be uniquely identified by each of the other devices in the system so that communication conflict is readily resolved between the various components within the analyte monitoring system 100.

In certain embodiments of the present disclosure, the sensor 101 is physically positioned in or on the body of a user whose analyte level is being monitored. The sensor 101 may be configured to continuously sample the analyte level of the user and convert the sampled analyte level into a corresponding data signal for transmission by the transmitter unit 102. In certain embodiments, the transmitter unit 102 may be physically coupled to the sensor 101 so that both devices are integrated in a single housing and positioned on the user's body. The transmitter unit 102 may perform data processing such as filtering and encoding on data signals and/or other functions, each of which corresponds to a sampled analyte level of the user, and in any event transmitter unit 102 transmits analyte information to the primary receiver unit 104 via the communication link 103. Additional detailed description of the continuous analyte monitoring system, its various components including the functional descriptions of the transmitter are provided in but not limited to U.S. Pat. Nos. 6,134,461, 6,175,752, 6,121,611, 6,560,471, and 6,746,582, and U.S. Patent Publication No. 2008/0278332 and elsewhere, the disclosures of each of which are incorporated by reference for all purposes.

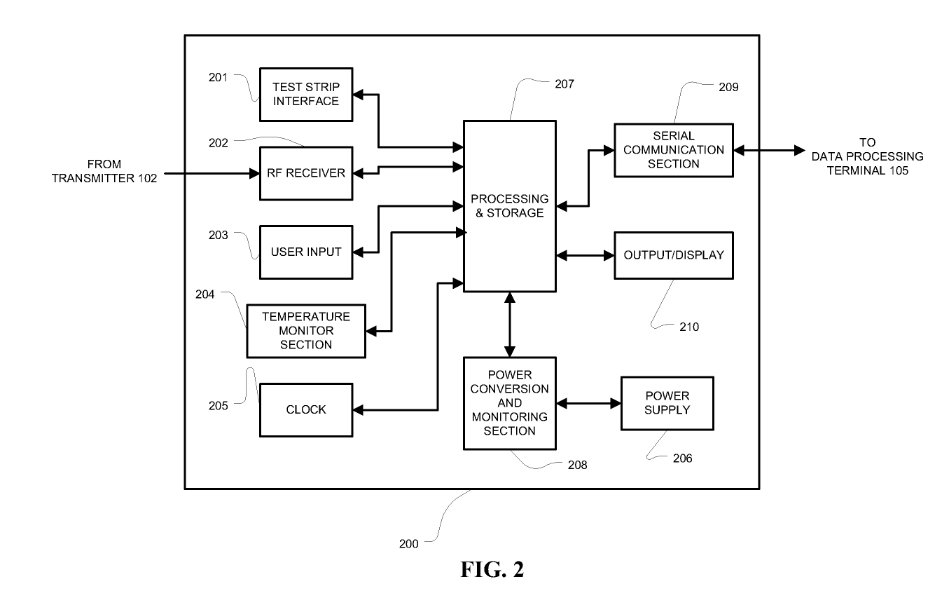

FIG. 2 is a block diagram of a receiver 200 according to embodiments of the present disclosure. In certain embodiments, receiver 200 may be the primary receiver unit 104 (FIG. 1) or the secondary receiver unit 106 as described above. As illustrated in the block diagram, the receiver 200 includes an analyte test strip interface 201, (e.g., blood glucose test strip port), a radio frequency (RF) receiver 202, a user input mechanism 203 (e.g., one or more keys of a keypad, a touch-sensitive screen, a voice-activated input command unit, one or more wheels, balls, buttons or dials, etc.), a temperature detection section 204, and a clock 205, each of which is operatively coupled to a receiver processor 207. In certain embodiments, the receiver 200 also includes a power supply 206, such as, for example, a rechargeable battery, operatively coupled to a power conversion and monitoring section 208. Further, the power conversion and monitoring section are also coupled to the receiver processor 207. A receiver serial communication section 209, and an output 210, such as, for example a display (e.g., a fill color organic light emitting diode (OLED) display, a liquid crystal display (LCD), a plasma display, etc.) or an audio speaker, are each operatively coupled to the receiver processor 207. In certain embodiments, the receiver 104/106 (FIG. 1) may include all or only some of the features of receiver 200 described in conjunction with FIG. 2.

In certain embodiments and as briefly discussed above, the analyte monitoring system 100 (FIG. 1) is a continuous glucose monitoring system, and the test strip interface 201 includes a glucose level testing portion to manually receive a glucose test strip to determine the glucose level of a blood sample applied to the test strip. In response to receiving a test strip, the receiver 200 may be configured to output blood glucose information determined from the test strip on the display. Additionally, the test strip can be used to calibrate a sensor such as, for example sensor 101. Accuracy of the measurement of the glucose information of the blood sample applied to a test strip and received and analyzed by the receiver 200 via test strip interface 201, is vital to the accuracy of the calibration of analyte monitoring system 100, in certain embodiments.

In certain embodiments, receiver 200 can be adapted to store information relating to a testing site from which a glucose (or other analyte) concentration level is measured from a biological fluid of a user, for example, the blood sample applied to a test strip and analyzed at the test strip interface 201. The testing site location could then be used to enhance calibration of analyte monitoring system 100. For example, during continuous glucose measurement (CGM) system calibration, sensor currents are paired with blood glucose readings to determine and/or update the sensor sensitivity which is used to calculate subsequent glucose readings. Typically, lag-correction is implemented to correct for blood-to-interstitial glucose dynamics to improve CGM accuracy.

Many CGM devices require that only fingerstick blood glucose readings be used for calibration. However, users may or may not be compliant with this requirement. Therefore, the CGM system could use the stored testing site location to modify the physiological or numerical model used to correct for blood-to-interstitial glucose lag based upon the source of the blood. In this manner, the stored testing site information can be utilized to correct blood to interstitial fluid analyte lag time. For example, if a fixed lag correction was used during calibration (e.g. if the blood glucose value is compared to the sensor reading at some future time, such as 5 or 10 minutes in the future) that fixed lag time could be modified to be appropriate for the blood to interstitial fluid glucose lag associated with particular blood glucose (e.g. finger or forearm) and interstitial fluid glucose (e.g. abdomen or back-of-the-arm) test sites. Additionally, calibration of sensor sensitivity may be improved, as described below. For example, if an appropriate estimate for the blood-to-interstitial glucose lag time was known, based upon the particular blood glucose and interstitial fluid glucose test sites, that information could be used to improve the sensor calibration such that the calibrated sensor reading could be scaled to more closely correlated with blood glucose values (e.g. venous blood glucose values).

In accordance with certain aspects of the present disclosure, receiver 200 can be configured to enable the user to input the testing site as part of a protocol to a blood glucose reading or other analyte reading. For example but not limitation, the testing site or location can include a fingerstick or an alternative site testing (“AST”) such as but not limited to, a hand, palm, arm, abdomen, thigh, or calf. In a similar manner, the receiver can be configured to allow the user to indicate that a reference analyte reading was obtained from a fluid other than blood, such as, but not limited to, saliva, sputum, conjunctival fluid, or urine.

Analyte measurement systems that allow for sample extraction from sites other than the finger and that can operate using small samples of blood, have been developed. For example, U.S. Pat. No. 6,120,676, the disclosure of which is incorporated herein by reference for all purposes, describes devices that permit generally accurate electrochemical analysis of an analyte, such as glucose, in a small sample volume of blood. Typically, less than about one μL of sample is required for the proper operation of these devices, which enables glucose testing through “arm sticks” rather than finger sticks. Additionally, commercial products for measuring glucose levels in blood that is extracted from sites other than the finger have been introduced, such as the Freestyle® blood glucose-monitoring system developed by Abbott Diabetes Care Inc., Alameda, Calif. Thus, in one aspect of the disclosure, blood assays can comprise less than or equal to about 1 μL of blood, such as for example, 0.5, 0.2 or 0.1 μL of blood sample or less.

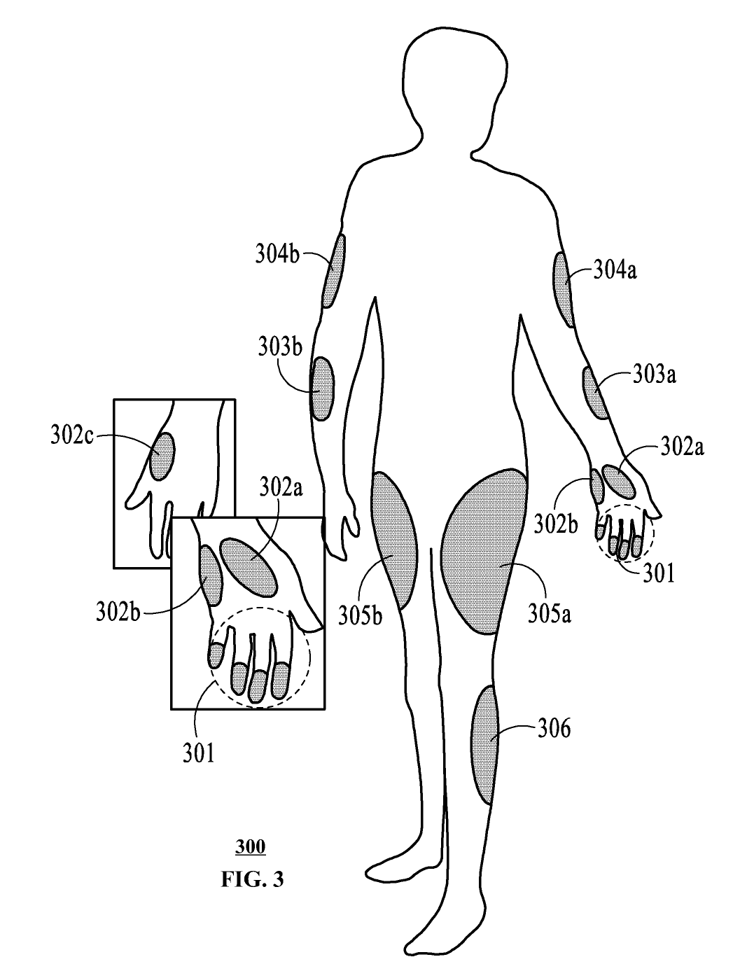

In this regard, receiver 200 may include a database of usable testing sites/locations and features to allow a user to input the testing site information via, for example, one or more input units 203. In one embodiment, the testing site information may be input to the receiver 200 via a touch screen. The touch screen may include a graphical representation, shown in FIG. 3, of a silhouette or physiological model of a user 300, with touch sensitive areas on the silhouette 300 corresponding to the testing site in use. Such touch sensitive areas may include, but are not limited to, a user's fingers 301 (for a fingerstick test), palm 302a/302b, hand 302c, forearm 303a/303b, upper arm 304a/304b, thigh 305a/305b, or lower leg area 306. In other embodiments, the user may be able to select the corresponding testing site via utilizing a button, wheel, trackball, touchpad, or joystick, and scrolling through a list of available testing site locations, which may be displayed as a text list (which may include corresponding check boxes, radial buttons, etc.) or as highlighted areas of silhouette 300. In still other embodiments, the user may be able to select a testing site by inputting a code or name of the testing site, such as by typing ‘finger’ (to correspond to a fingerstick testing site) into a keyboard provided on or connected to receiver 200. In other embodiments, receiver 200 may include a microphone and voice recognition software, such that a user can say the testing site being utilized and receiver 200 can automatically select the corresponding site from the database. It is also contemplated that combinations of the above methods may also be employed for selecting the testing site.

As described above, analyte, such as glucose, measurements may vary based on the site of an in vitro test, which may be used for calibration of a CGM system. Such variations may be due to, but are not limited to, time lag as described above, glucose concentration level, and effect of interferents. For example, the time lag between a CGM measured glucose concentration and a blood glucose measurement taken from a finger may be different than a CGM measured glucose concentration and a blood glucose measurement taken from a forearm measurement, such that, for example, a lag between CGM measured glucose concentration and a fingerstick blood glucose measurement may be approximately 2-20 minutes, while a lag between a CGM measured glucose concentration and a forearm blood glucose measurement may be approximately 5-30 minutes. Further, for example, time lag between a CGM measured glucose concentration and a thigh blood glucose measurement may be approximately 15-40 minutes. The preceding estimated lag time durations are exemplary only, and accordingly, shorter or longer lag times for different body areas are also included within the scope of the present disclosure. Furthermore, the rate of change of the analyte concentration such as glucose fluctuation may affect the time lag between a CGM measured glucose concentration compared with an in vitro blood glucose measurement.

As described above, different locations on the body may also have an effect on the overall glucose concentration measured. For example, a fingerstick test may have a reading of 100 mg/dL, while a time corresponding measurement from forearm may be 97 mg/dL, and a time corresponding measurement from thigh may be 95 mg/dL. In certain embodiments, the different measurement results based on the different body site are obtained when the fluctuation of glucose level is minimal—that is, when the glucose concentration is substantially stable such that the rate of change of glucose is near zero. Accordingly, in certain embodiments, locations of the reference measurement source such as fingertip, thigh, or forearm may be provided in conjunction with the calibration algorithms of analyte monitoring system 100 to improve accuracy of the CGM systems. It is to be noted that the above is for example purposes only, and that differences in glucose readings between sites may be more or less than indicated, including no difference. Further, in certain embodiments, different body sites may have different effects from interferents in the blood. For example, a fingerstick test may have a lower or higher correction factor for interferents than a forearm or thigh test.

In certain embodiments, analyte monitoring system 100 may be trainable, programmable or programmed to learn from past data or user behavior as provided to the system. For example, receiver 200 may include programming, such as calibration and lag correction algorithms, corresponding to varying testing sites on a user's body. These algorithms may be pre-programmed, or in other embodiments, may be programmed by the user or a medical professional. Analyte monitoring system 100 may store historical analyte related data, for example in memory 207 of receiver 200, and utilize the stored historical data to modify the calibration and correction parameters, such as lag correction parameters. Accordingly, the calibration and other correction factors can be customized for the user over time. Further, receiver 200 may store usage data, such that when a user primarily utilizes a particular testing site, such as a finger, the primary testing site is used as the default testing site when choosing a testing site. Alternatively, the default testing site may be pre-programmed. In other embodiments, no default testing site is used.

In certain embodiments, receiver 200 may include a database of usable testing sites for not only in vitro calibration tests, but also usable placement sites for a continuous glucose monitoring system measuring glucose levels based on an interstitial fluid measurement. Similar to the silhouette 300 of FIG. 3, a menu of the receiver 200 may include a silhouette, or text or other visual list, of usable sites for a CGM system. Accordingly, when a CGM system is placed at one of the usable sites and activated for use, the user may choose the appropriate site from the silhouette or list. Each usable site may correspond to various factors, including time lag, concentration level, interferent effect of the site, and skin thickness, as described above. These factors may then be applied to glucose level calculations and calibration calculations, such that the accuracy of all data analysis is optimized. Further, a similar silhouette or list may be utilized for choosing an appropriate site for an infusion set for use with an insulin pump or other insulin or other medication administration (e.g., insulin pen, single dose injector), if used by the user. This may allow a therapy calculation feature of the CGM system to accurately recommend an insulin amount or regiment based on the effect of the insulin based on the site of the administration (e.g., the time taken for the insulin to lower the blood glucose level, insulin absorption rate, etc.). In certain embodiments, the type of insulin, such as fact acting or long acting, may also be entered and taken into account to further achieve an optimal insulin dosage.

In certain embodiments, more than one analyte sensor may be used by a user. In such embodiments, a similar silhouette to that of silhouette 300 may be shown on the receiver, such that a user can specify the location of each of the analyte sensors.

In certain embodiments, other methods of obtaining accurate calibration results may be employed in addition to, or separately from, utilizing testing site information for accurate calibration. For example, improved two point (or more) calibration systems are disclosed. In certain embodiments, with respect to glucose monitoring systems, fluctuations in glucose levels may be utilized to calibrate a glucose analyte monitoring system, such as for example, continuous glucose monitor or on-demand glucose monitor systems. For example, when the analyte monitoring device detects either a low or high concentration value, such as an elevated value (hyperglycemia) or a depressed value (hypoglycemia), the system can prompt the user to assay a blood sample to confirm the high or low analyte levels. The blood assay can be used for a system calibration. In some embodiments, if the blood assay occurs within a window of time (e.g. within 0 to 2 hours) of a scheduled calibration time, that assay can be used as a calibration attempt and the scheduled request for calibration can be skipped. For example, the user can be prompted to perform a blood assay, such as by way of a fingerstick, to confirm high or low glucose alarm. The fingerstick can be used for system calibration.

Additionally, as described below, the weight of the fingersticks for system calibration can be determined based upon the system's assessment of the reliability of the fingerstick. For example, if a continuous glucose measurement reading is 70 mg/dL and the fingerstick is 74 mg/dL, the analyte measurement system determines that the fingerstick is highly reliable and the system would heavily weight the fingerstick in an update of the system calibration. Alternatively, if the continuous glucose measurement reading is 70 mg/dL and the fingerstick is 94 mg/dL, less weight could be assigned to that fingerstick in an update of the system calibration.

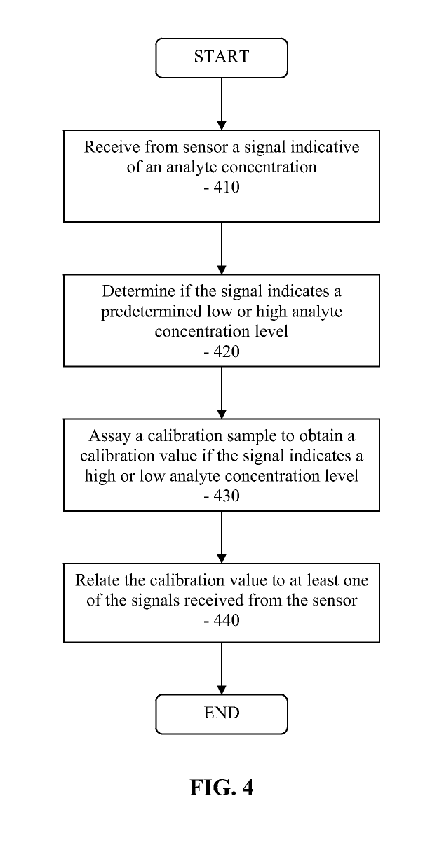

In one embodiment, as shown in the flow chart of FIG. 4, a method of calibration may include the steps of receiving a signal from the sensor, the signal corresponding to an analyte concentration level in a biofluid of a user (410), determining if the signal indicates a predetermined low or high analyte concentration level (420), prompting the user to assay a calibration sample of the user's blood to obtain a calibration value, if the signal indicates a high or low analyte concentration level (430), and relating the calibration value to at least one of the signals from the sensor (440). In certain embodiments, the analyte may be glucose, and the high and low analyte concentrations levels are within a normal range, such as a euglycemic range.

In certain embodiments, the method can be employed with a one-point calibration system. For example, in one embodiment, the method could be employed with a one-point calibration system wherein the system prompts a user for a calibration attempt when the analyte level, as determined by the signal from the sensor, reaches a predetermined high range. At this high range, the signal-to-noise ratio would be expected to be lower such that an improved accuracy of calibration may be obtained. The one point reference data for calibration can correspond to an elevated analyte range, such as in a hyperglycemic range, or alternatively, the one point data can correspond to a depressed analyte range, such as in a hypoglycemic range. For example, the reference data or blood assay can exhibit analyte levels above or below for example 60 to 350 mg/dL.

In certain embodiments, the method can include the steps of determining whether the prompted assay is within a window of time for a prescheduled calibration prompt and skipping the prescheduled calibration prompt if the prompted assay is indeed within the window. For example, the window of time may be three hours or less. Where the analyte measurement system includes prescheduled calibration times, and the unscheduled blood assay is used as the calibration point, the calibration prompt can be reset to occur at a time in the future.

As described above, the assayed calibration sample can be obtained from a fingerstick testing site, or alternatively, an alternative site test. In this manner, the method can include the step of storing the testing site location, as described above.

In some embodiments, a predetermined low or high analyte concentration level can be calculated based upon a percentage of a user's average analyte level. This allows the determination of “high” and “low” ranges using an uncalibrated sensor.

The calibration value can be compared to at least one signal from the sensor for use in calibrating the sensor. In some instances, the calibration value is discarded if it is not within a predefined threshold of the at least one of the signals from the sensor. This could be used, for example, as an outlier check to indicate if the reference value (e.g. fingerstick) is likely an error, or as a check on the quality of the sensor signal (e.g. early signal attenuation (ESA) check or sensitivity check). Further description of outlier check and ESA is described in, among others, U.S. patent application Ser. Nos. 12/152,648, 11/925,689, and 12/362,475, the disclosures of each of which are incorporated herein by reference for all purposes.

In some embodiments, the calibration value can be weighted based upon the difference between the calibration value of the assayed sample and the signals from the sensor. In this manner, the calibration value is discarded if the absolute value of the rate of change of the current analyte value exceeds a threshold value because of the potential lag between the actual analyte value and the sensed analyte value. For example, if the analyte is glucose, there can be a lag between blood glucose and interstitial glucose. For example, if blood glucose is changing at a rate of 3 mg/dl/min, and there may be a 10-minute lag between blood and interstitial fluid glucose levels, a bias of 30 mg/dL may be imparted into the calibrated sensor glucose reading, with the direction of the bias depending on the direction of change in glucose. If rates of change are lower, for example, if blood glucose is changing at a rate of 0.25 mg/dl/min and there is a 10-minute lag between blood and ISF glucose levels, calibration might only impart a bias of 2.5 mg/dL in a calibrated sensor glucose reading. Lag correction approaches can minimize these errors. However, it is preferable to calibrate during times of stable glucose values.

In one embodiment, prescheduled system calibrations can be weighted differently based upon their distance from either the user's average glucose or from the glucose level at which previous calibrations have occurred. This approach could be easily extended to the weighting of these “opportunistic” calibrations by assigning more weight to calibration attempts that have a higher confidence. For example, in the case of glucose, if the sensor glucose level reads 72 mg/dL and the fingerstick blood glucose level reads 74 mg/dL, there would be a high confidence that the fingerstick is accurate and would be a good candidate to be used for calibration. As such, it could be weighted as 100% or 90% or 70% (with respect to previous calibration attempts or factory calibration assignments) in the determination of sensor sensitivity. Similarly, these weightings could also be extended to these opportunistic calibrations, where the weighting could be increased if the calibration is farther from (e.g. greater than) either average glucose values or from values at which previous calibrations occurred. Additional description of calibration routines can be found in US Patent Publication No. 2010/0274515, the disclosure of which is incorporated by reference for all purposes.

Acceptance of each calibration point could be subject to conditions, such as that the glucose rate of change absolute value must not exceed a threshold value or these points could also be subject to corrections, such as lag correction. For example, pseudo-retrospective (lag correction) calibration approaches could easily be incorporated into this approach. Pseudo retrospective calibration, as used herein, refers to a comparison between a reference (e.g. blood glucose) value from some time t=0 with a sensor (glucose) value from some time t=+X in order to help account for the lag between reference (blood glucose) and sensor (interstitial fluid glucose) values. As such, the weighting of opportunistic calibrations can be independent of these approaches. Following this approach, the fingersticks would be more likely increase calibration accuracy and the risk of introducing error from a single poor calibration could be minimized.

In accordance with the single point calibration described above, the method can include the step of interpreting the one point calibration as a two point calibration where the second point is assumed to be zero. Accordingly, the general concept of maximum separation of calibration points in order to improve accuracy still applies.

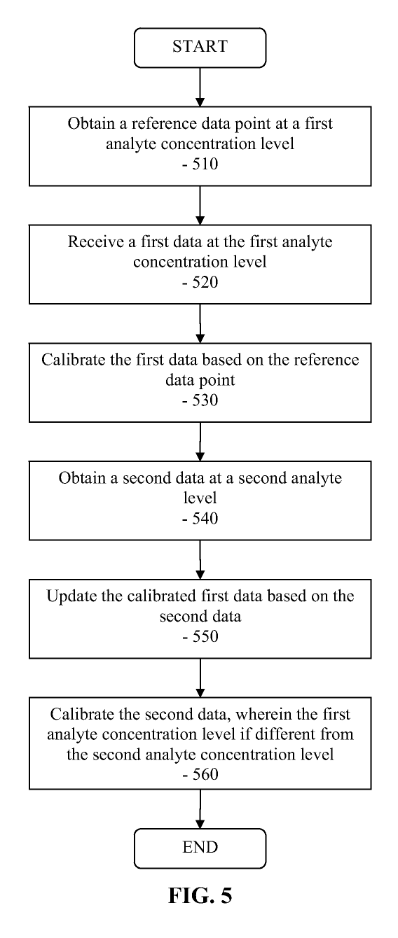

In another embodiment, a two point or more calibration is provided as shown in FIG. 5. For example, the method includes obtaining a reference data point at a first analyte concentration level (510), receiving a first data at the first analyte concentration level (520), calibrating the first data based on the reference data point (530), obtaining a second data at a second analyte level (540), updating the calibrated first data based on the second data (550), and calibrating the second data, wherein the first analyte concentration level if different from the second analyte concentration level (560). In this regard, the calibration accuracy is improved when the calibration points or reference data points are different, the more different the two points, the more accurate the calibration. For the purpose of illustration, a two point calibration with a first reference point of 100 mg/dL and a second reference point of 120 mg/dL would be less accurate than a two point calibration with a first reference point of 40 mg/dL and a second reference point of 400 mg/dL.

In certain embodiments, analyte monitoring system 100 may ignore or postpone calibration when a first and second reference analyte concentrations received for calibration are not sufficiently different. For example, as described above, a two point calibration with a first reference point of 100 mg/dL and a second reference point of 120 mg/dL, may be considered an inaccurate calibration. Accordingly, analyte monitoring system 100 may not use the second reference point for purposes of calibration and analyte monitoring system 100 may accordingly output a notification to the user that calibration did not occur, and further to wait a predetermined time period before obtaining another reference point which is then compared against the first reference point, for example, to determine if there is sufficient distance between the obtained reference point and the first reference point for purposes of calibration.

More specifically, when calibration is not performed due to a second calibration point being too close to a first calibration point, the analyte monitoring system 100 may notify the user to delay providing the next calibration sample. This is due to the fact that if a new calibration sample is taken immediately or substantially temporally close to the rejected calibration point/measurement, the new calibration measurement may still be too close to the first calibration measurement value, and thus not accepted for calibration. Accordingly, if a next calibration sample is not taken until after a predetermined wait period, such as 2 hours, for example, the probability of a varied calibration measurement increases, and thus the likelihood of a more accurate calibration can be increased. In such embodiments, a calibration schedule for the analyte monitoring system 100 may be further updated to reflect the change in calibration time.

Whether calibration measurements are deemed accurate based on a comparison with previous calibration measurement, may be based on a predetermined difference in analyte concentration. For example, in some embodiments, calibration measurements are considered acceptable or accurate when they are more than 50 mg/dL different than the preceding calibration measurement. In other embodiments, other ranges may be used, such as 20 mg/dL, 40 mg/dL, 60 mg/dL, 80 mg/dL, 100 mg/dL, 150 mg/dL or more or less. In certain embodiments the acceptable range is programmable and/or modifiable, such as by the user or a medical professional based on a user's personal analyte or glucose profile. In certain embodiments, the range may be adjusted automatically by the analyte monitoring system 100 by analyzing historical or past data and adjusting the range. In other embodiments, the range may vary based on time of day, time of month, or time of year.

In certain embodiments, analyte monitoring system 100 includes a calibration schedule for calibrating sensor 101. The calibration schedule may include requesting or prompting for a calibration sample at predetermined time intervals, such as every 12 hours, ever 24 hours, every 2 days, every week, etc. In other embodiments, the calibration schedule may include time intervals that very based on time of day, e.g., at certain times of the day, such as upon waking, before or after eating or exercising, before administering medication, or before sleeping. In other embodiments, the calibration schedule may be personalized to a user based on a historical personal profile. The calibration schedule may also include a combination of any of the above.

In certain embodiments, a user may take a manual analyte measurement outside of a predetermined calibration schedule, for example, just prior to administering a medication, such as insulin. Such a measurement may be used as a calibration measurement. In certain embodiments, if an unscheduled measurement is taken within a predetermined time of a scheduled calibration, the unscheduled measurement may be used as the calibration measurement, and no notification may be presented at the time of the next scheduled calibration. In certain embodiments, whether the unscheduled calibration measurement will replace the upcoming scheduled calibration, may depend upon the value of the unscheduled measurement. For example, the unscheduled measurement value may be compared to a previous calibration measurement to determine whether the two measurements sufficiently differ to allow for accurate calibration. If the unscheduled measurement does not sufficiently differ from the previous calibration measurement, the confidence level of the unscheduled measurement may not meet a predetermined threshold acceptability level, and the analyte monitoring system 100 may be programmed to not adjust the calibration schedule to reflect the unscheduled measurement, simply log or store the unscheduled measurement but otherwise, ignore it for purposes of calibration, and remain with the scheduled calibration.

To perform calibration based on discrete measurements, in certain embodiments, analyte monitoring system 100 may employ a substantial plurality of signal processing algorithms, which may be performed by transmitter 102 and/or receiver 104/106, or a combination thereof. Over the usable life of sensor 101, calibrations may be performed at various intervals in order to determine that the sensor is ready for use and continues to operate in a useful range, and to determine the sensitivity of the sensor so that accurate analyte concentration measurements may be provided.

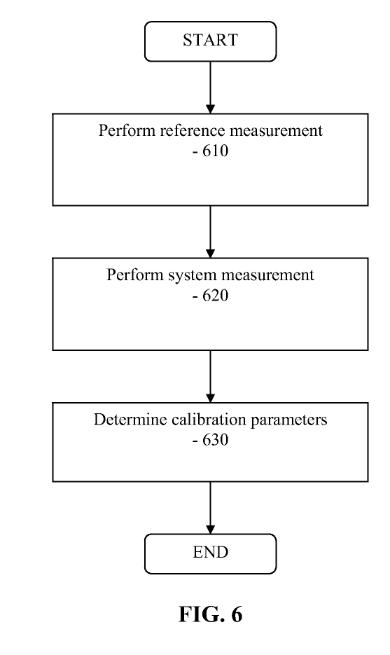

FIG. 6 shows an exemplary procedure for calibrating an analyte monitoring system, such as system 100. In general, such a procedure may comprise taking a discrete analyte measurement from the subject (“reference measurement” 610), taking at a proximate time an analyte measurement from the subject with system 100 (“system measurement” 620), and determining, based on such measurements, an appropriate calibration or sensitivity factor (5) for converting system measurements into concentration units (630). The reference measurement may be a blood glucose fingerstick (in the case of the analyte being glucose), but also may be any measurement of analyte in the subject, blood-based or otherwise, taken by any means other than the system being calibrated.

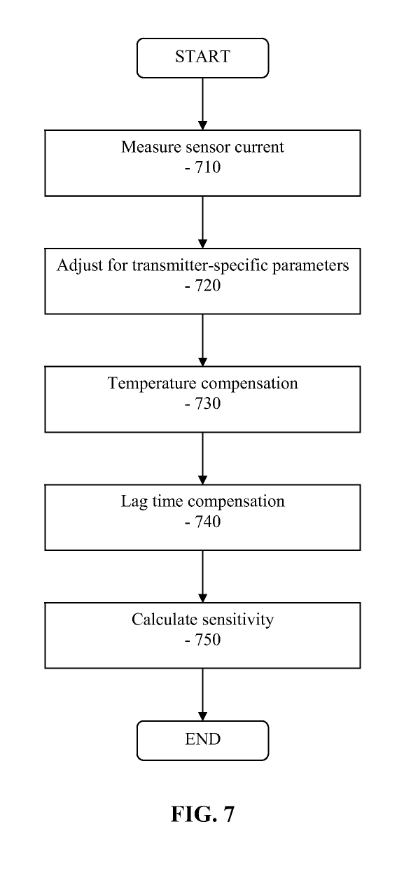

A procedure for taking a system measurement in certain embodiments is outlined in FIG. 7. The procedure may generally comprise a measurement taken from sensor 101 (710), which is processed by transmitter unit 102, receiver unit 104/106 or data processing terminal 105. In some embodiments, the measurement from sensor 101 may be an electrical current signal. Transmitters may vary from one to another in terms of electrical and physical characteristics. Accordingly, the sensor current measurement may be adjusted for variations among transmitters in accordance with parameters that characterize the particular transmitter 102 in use (720). The current may then be further subjected to temperature compensation (730) and, if sufficient data is available, lag time compensation (740), the latter being applied due to the delay in interstitial analyte concentration measurements as compared to discrete blood measurements, when the analyte level is changing. An “immediate, real-time” sensitivity factor may be calculated (750) by dividing the temperature and lag-corrected sensor current divided the reference measurement (each determined at appropriate times). Furthermore, a composite sensitivity may be calculated based on successive measurements, for example, two successive measurements, by performing a weighted average of the sensitivities calculated from the two measurements.

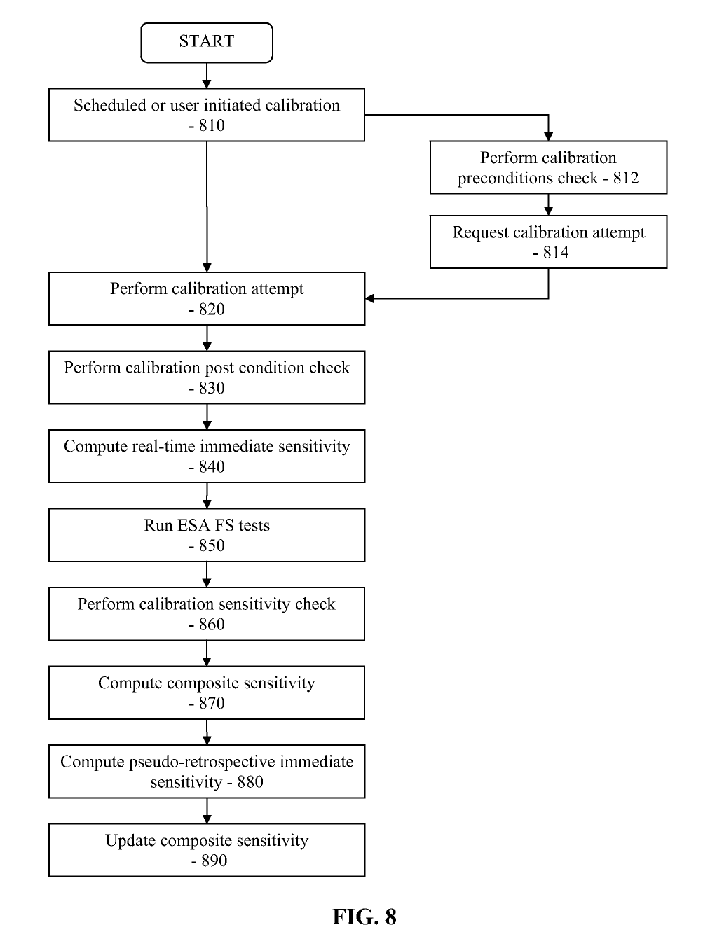

FIG. 8 is a flow diagram that outlines in further detail a number of phases for a calibration procedure in certain embodiments of the disclosed subject matter, particularly developed for continuous monitoring embodiments.

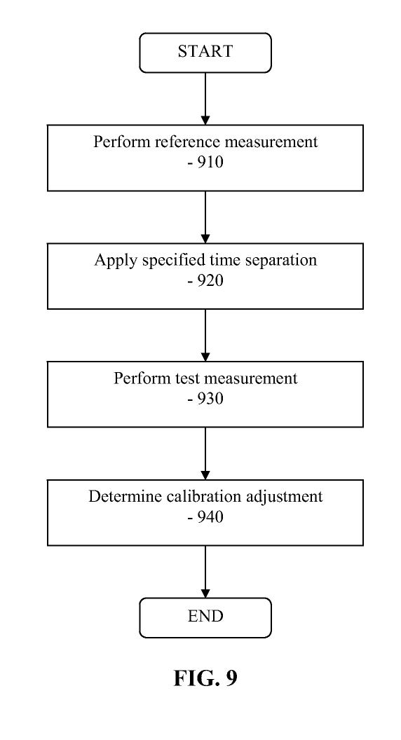

In an on-demand system, certain adaptations will be introduced into the processing described in connection with FIG. 8, as well as in connection with FIG. 9, which follows. As will be seen, there are numerous calculations performed in connection with FIGS. 8 and 9 that contemplate a series of periodic or intermittent system measurements, as would normally be obtained during the operation of a CGM device. However, in an on-demand device, the transmitter unit may communicate on separate, relatively widely spaced occasions, with the receiver unit. Various techniques may be used to acquire, in an on-demand setting, the series of measurements contemplated by FIGS. 8 and 9, or to work around not having some or all of such data. For example, in embodiments in which the transmitter includes storage for recent measurements, an on-demand calibration may invoke a bulk transfer of stored values, which may be sufficient to satisfy the requirements of the procedures envisioned by FIGS. 8 and 9. In other embodiments, the transmitter may provide averaged and sequential data that may be used in a similar manner, although the sequenced data may provide fewer data points than might be used in a CGM counterpart performing the same procedures, the procedures could be performed with the fewer number of points. The transmitter could also provide rate of change measurements, e.g., by a differentiator circuit, or by comparison to a running average. Similarly, “retrospective” adjustments, as will be discussed, requiring a series of system measurements after a calibration, could similarly be provided by a follow-up on-demand measurement within a specified period of time. In addition, in some embodiments, e.g., where such data is not available, the calculations could proceed without the sequential data, using the last data acquired in place of an average, or not adjusting for rates of change where insufficient data is available to calculate those rates. A number of specific embodiments for acquiring periodic, averaged and/or rate-of-change measurements in an on-demand context are discussed later on in this disclosure.

With the foregoing in mind, with regard to inherent differences between CGM and on-demand operating characteristics, the routine shown in FIG. 8 is now described in further detail, with reference to certain embodiments.

The calibration process in these embodiments begins at step 810, with either a scheduled or user-initiated calibration. In these embodiments, system 100 expects calibration when either a scheduled calibration is due, or the user indicates intent to perform manual calibration, for example, by appropriate input into a CGM monitor, or alternatively by initiating an on-demand measurement.

The electrical current produced by analyte sensor 101, the temperature of the skin near the sensor, and the temperature of the circuitry may be checked for validity within transmitter 102. Whenever transmitter 102 is connected to receiver unit 104/106 (whether on a “continuous” basis or in an on-demand connection), these measurements and checks are transmitted to the receiver unit. In some embodiments, transmitter 102 transmits data to receiver unit 104/106 via a “rolling data” field in a periodic data packet. Data may be spread out among consecutive data packets, and the packets may provide redundancy (and further reliability and data integrity) by accompanying current values with immediate past values. Other embodiments, e.g., in which the transmitter collects logged and/or time-delayed data, may transfer larger amounts of data with each transmission, examples of which may be found in, among others, U.S. patent application Ser. No. 12/807,278, the disclosure of which is incorporated herein by reference in its entirety for all purposes. Data transmitted may include measurement calibration information and a “count” of the sensor measurement from an analog to digital converter (ADC).

After a calibration is initiated per step 810, a calibration preconditions check 812 may be performed. In one embodiment, these checks may include data validation on the transmitter side, including checks for hardware error (a composite OR of a plurality of possible error signals), data quality (set if the sensor measurement is changing faster than could be accounted for physiologically, indicative of an intermittent connection or leakage) and current/voltage saturation (compared to current and voltage thresholds). If any of these conditions are detected and then cleared, the corresponding flag bit remains set for a period, e.g., one minute, after the condition clears, to give time for the system to settle. Further checks may be performed within receiver 104/106. A counter electrode voltage signal may be checked to ensure that it is within operating range, and if not the receiver processor may set a flag for invalid data not to be used for measurements (and hold the flag for a period, e.g., one minute, after the condition clears).

A data quality check may further comprise checks that all requisite data has been supplied by the transmitter, that none of the various error flags are set, and that the current and prior voltage counts were within prescribed limits (e.g., about 50-2900 voltage counts). There may be further validation that the transmitter temperature is in a valid range (e.g., about 25-40° C.), that raw sensor current is above an acceptable threshold (e.g., about 18 counts), and that sensor life state is still active. There may also be a further check for high-frequency noise.

A data availability check may also be performed. In this check, after eliminating points marked as invalid per the above-described processes, as well as those invalidated by upstream processes, a determination is made whether there are sufficient valid data points to reliably perform rate-related calculations, as may be required in various aspects of the calibration procedure. The data availability check may be varied for on-demand applications: they may be based on an examination of stored data received in the latest transmission (where the transmitter stores data or provides time-delayed data), or alternatively, these tests could be reduced or eliminated. A minimum wait requirement check may be performed, to ensure that the calibration request does not conflict with the operative calibration schedule. As will be discussed, calibration scheduling imposes limitations on when calibrations may be taken and/or used, including waiting periods during baseline calibrations and at certain other times.

A sensor rate check may also be performed. A rate is calculated from a plurality of measurement points, based on a least-squares straight-line fit, again, where data is available. The value of the rate thus established must be less than the composite sensitivity (or if not yet calculated, a nominal sensitivity), multiplied by the sensor current. Pre-calibration check procedures are further discussed in, among others, US publication nos. 2008/0161666 and 2009/0036747, the disclosures of each of which are incorporated by reference herein in their entirety for all purposes.

If conditions permit (or require) calibration, and calibration is called for or expected in accordance with a calibration schedule, or user initiated, a calibration attempt may be requested 814. Calibration “attempt” for purposes herein refers to a reference measurement used or evaluated for calibration purposes. In some embodiments, requesting a calibration attempt comprises providing a prompt, for example, through a screen on receiver 104/106, or an audible prompt, to take a reference measurement, e.g., a BG fingerstick. Pursuant to request (814), a reference measurement is taken for calibration purposes and a calibration is attempted 820.

After the user has conducted a reference measurement for calibration, further checks may be performed 830, to check the sensor condition since the request for the reference test was made, and to ensure that the reference measurement is within an acceptable range. Such check may further comprise the same tests as the pre-calibration checks, except that user interaction delays will not be factored into rate windows and determinations, and scheduling wait time constraints will not be considered (since the calibration has already started). Post-calibration check procedures are further discussed in, among others, US Patent Publication Nos. 2008/0161666 and 2009/0036747, the disclosures of each of which are incorporated by reference herein in their entirety for all purposes.

After reference test data is acquired and checked, sensor sensitivity may be determined 840. Measured sensor sensitivity may be affected by a number of factors, for which appropriate corrections may be introduced, including temperature and lag corrections.

As mentioned above in connection with FIG. 7, sensor sensitivity is also temperature dependent. The measurement of skin temperature can be influenced by the temperature of the environment around the transmitter case. To account for this dependence, system 100 may use two thermistors, one in the skin, and the other in the transmitter circuitry, to measure these temperatures, and then compensate. A lag adjustment may also be calculated. In comparing a measured interstitial analyte measurement with a blood-derived reference measurement, in a subject whose analyte level may be changing, there could be a time lag of the interstitial measurement as compared to the blood-based reference measurement, which could affect the accuracy of the calibration unless appropriately taken into account. In one embodiment, the lag corrected monitored data at the calibration time may be determined by applying the determined rate of change of the monitored data at the calibration time to a predetermined constant value. In one embodiment, the predetermined constant value may include, a predetermined time constant. For example, in one embodiment, the predetermined time constant may include a fixed time constant in the range of approximately four to fifteen minutes, and which may be associated with the one or more of the patient physiological profile, one or more attributes associated with the monitoring system (including, for example but not limited to, the characteristics of the analyte sensor 101). In a further aspect, the predetermined time constant may vary based on one or more factors including, for example, but not limited to the timing and amount of food intake by the patient, exogenous insulin intake, physical activities by the patient such as exercise, or any other factors that may affect the time constant, and which may be empirically determined, examples of which can be found in, among others, US publication no. 2008/0081977, the disclosure of which is incorporated herein by reference in its entirety for all purposes.

Certain embodiments employ one or more procedures to detect early signal attenuation (ESA) 850 to avoid giving inaccurate readings while the system is in an ESA condition. Early signal Attenuation (ESA) refers to a condition in which the effective sensitivity of a sensor appears to attenuate and then recover in the early stages of the sensor life. For example, for some insertions, the sensitivity of the system may be attenuated during the first 24 hours after insertion. The states that may be defined with respect to ESA, and the transitions between those states, are discussed below in connection with calibration scheduling. As will be further addressed in that discussion, ESA detection may be performed, in some embodiments, primarily during periods in which ESA is likely to occur, e.g., within the first 24 hours after insertion. ESA detection procedures are further described in, among others, U.S. patent application Ser. No. 12/363,712, the disclosure of which is incorporated herein by reference in its entirety for all purposes.

In certain embodiments, two calibration sensitivity tests 860 are performed, after passing the ESA tests described above: an absolute test, and a relative (outlier) test. In the absolute sensitivity test, the measured immediate sensitivity compared to the nominal sensitivity for the sensor. The relative sensitivity test is intended to eliminate “outlier” measurements from being used to calculate composite sensitivity. As will be discussed, a composite sensitivity calculation, in some embodiments, requires two sensitivity figures, S1 and S2. Sᵢ₍ₖ₎, Sᵢ₍ₖ₋₁₎, and Sᵢ₍ₘ₎ are chosen as discussed previously, in connection with ESA. If Sᵢ₍ₖ₎/S₍ₖ₋₁₎ (e.g., current valid sensitivity compared to preceding valid sensitivity) is in the range of about 0.778 to 1.5, then Sᵢ₍ₖ₋₁₎ (the prior value) will be used as S1, and Sᵢ₍ₖ₎ (the current value) will be used as S2. If the foregoing test fails, then, if there is an S(m) established, and if Sᵢ₍ₖ₎ compared to the previously determined composite sensitivity (Sc) falls within the above range, then Sᵢ₍ₘ₎ will be used for S1 and Sᵢ₍ₖ₎ will be used as S2. Otherwise, another calibration attempt is requested, for which Sᵢ₍ₖ₎ will become Sᵢ₍ₖ₋₁₎, and as part of the new determination, the relative (outlier) test will be repeated.

After the calibration sensitivity check 860, in certain embodiments, a composite sensitivity test is performed 870. The composite sensitivity, S_{c}, is used to convert sensor current in units of ADC counts to calibrated analyte (e.g., glucose) in units of mg/dl in some embodiments. For the first calibration, the “composite” sensitivity is equal to the sensitivity from a single valid calibration attempt. When appropriate thereafter, multiple sensitivities are used to determine the composite sensitivity. For the first calibration, the composite sensitivity takes the value of Sᵢ₍ₖ₎. Afterwards, the composite sensitivity is a weighted average of the S₁ and S₂ values determined by the outlier check:

S_{c}(k)=S₁W₁+S₂W₂

The first weighting parameter and the second weighted parameter may be different or substantially equal. They may, for example, be one or both of time based, or based on a prior calibration parameter. In certain embodiments, the weighing factors used are about 0.4, 0.42, 0.433, 0.444, etc. for W₁, and 0.6, 0.58, 0.567, 0.556, etc. for W₂. In some embodiments, the weighting factors may depend upon when the analyte measurement was taken, e.g., more recent analyte measurements may be assigned a larger weighting factor.

S_{c} may need to be updated between calibrations, as a result of a pseudo-retrospective immediate sensitivity adjustment, in which case S₂ will be replaced with a new value from that adjustment.

During operation of the receiver, a calibrated analyte concentration figure (G_{CAL}) may be obtained using the currently valid composite sensitivity:

G_{CAL}=G_{rTC}/S_{S}_{C}

The latest immediate sensitivity value S₂ used to calculate composite sensitivity incorporates, as discussed, a lag correction to take into account the delay between a change in blood analyte level and a corresponding change in the interstitial level of the analyte. However, if analyte levels continue to change after a calibration, it may be possible, in some embodiments, to improve the lag correction by factoring in system measurements taken after the calibration, and use the improved correction to update S₂, and, correspondingly, S_{c}. This correction is based on subsequent system measurements, and accordingly may be done without taking a new reference measurement (e.g., fingerstick).

In certain embodiments, this correction, referred to as a pseudo-retrospective immediate sensitivity correction 880, is calculated after about seven system measurements have been taken after the prior calibration (provided no subsequent calibration attempt becomes eligible for update before this number of system measurements have been collected), of which at least about four are valid. Alternatively, the retrospective data could be provided by a subsequent on-demand system measurement. If the standard error associated with computing the adjusted analyte count is less than the standard error in the underlying lag correction calculation (e.g., an improved correction is indicated), the sensitivity used for S₂ may be updated accordingly.

To perform the correction, a new least-squares fitted line may be determined, taking into account the additional post-calibration data system measurements, and the slope (rate) and intercept of this line used to calculate a corrected value (G_{PrLrTC}) for the real time value G_{RtLrTC}, which may be divided by the reference measurement from the latest attempt to obtain an updates sensitivity to use as S₂. These procedures for calculating a pseudo-retrospective immediate sensitivity correction are further described in, among others, US publication no. 2008/0081977, the disclosure of which is incorporated herein by reference in its entirety for all purposes.

As noted, if a pseudo-retrospective immediate sensitivity correction is performed, resulting in an updated value for S₂, then a corresponding update composite sensitivity factor, S_{c}, may be calculated 890. The value of S₁ used in the earlier calculation of S_{c} will continue to be used.

Further description of the procedures outlined in FIG. 8 can be found in, among others, US Patent Publication Nos. 2009/0005665; 2008/0288204; 2009/0006034; 2008/0255808; 2008/0256048; 2009/0006034; 2008/0312842; 2008/0312845; 2008/0312844; 2008/0255434; 2008/0287763; 2008/0281179; 2008/0288180; 2009/0033482; /2008-0255437; and 2009/0036760, the disclosures of each of which are incorporated herein by reference for all purposes.

On-demand monitors will generally not automatically perform system measurements after a discrete calibration attempt, because such monitors inherently rely on the user to initiate a system measurement, e.g., by bringing the receiver into proximity of the transmitter and/or providing a user input, such as a pressing a button. Referring to FIG. 9, an adapted calibration approach that may be used with an on-demand monitor is described.

The system in certain embodiments provides a reference measurement of a level of said analyte in the subject to be performed by a method other than use of the system being calibrated (910). The system causes the user to use the on-demand system to perform at least one test measurement of a level of said analyte (930), within about a specified period before or after the time of the reference measurement (920). The system determines a calibration adjustment, as a function of at least said reference measurement and said at least one test measurement (940). The reference measurement in the foregoing protocol could be caused to be conducted at a time in accordance with a calibration schedule for the on-demand device.

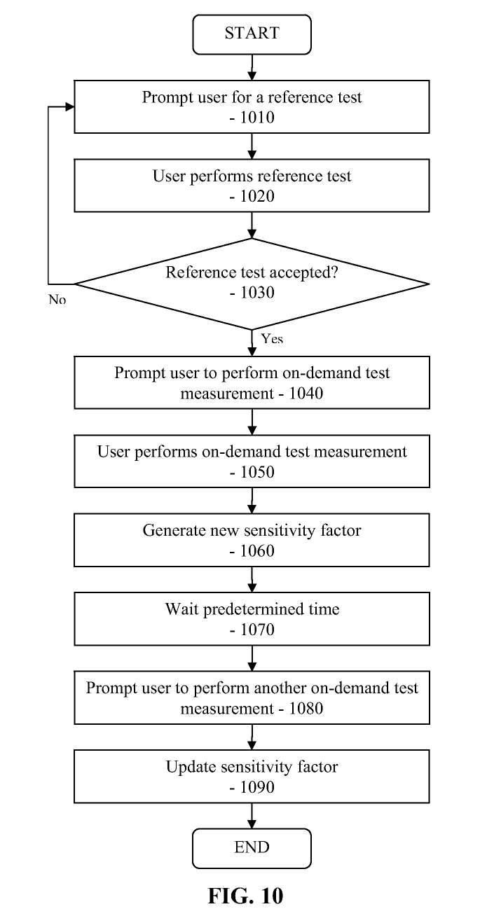

In certain embodiments, for calibration schemes where sensor data prior to and substantially proximate to the calibration BG reading are used in the sensitivity calculation, a more detailed adapted calibration procedure could be as shown in FIG. 10. The receiver unit may prompt the user for a reference test (1010). The user then performs a reference measurement (1020). If the calibration logic in the receiver accepts the reference measurement for calibration (1030), then the receiver unit may prompt the user to acquire an “on-demand” test result with the device (1040). The user then performs the on-demand test measurement, e.g., by bringing the receiver unit into proximity with the transmitter device so as to induce a test measurement to be taken (1050). The receiver unit processes the reference measurement and test measurement taken on demand to generate a new sensitivity factor for calibration of the system (1060).

The foregoing procedure differs from a CGM calibration procedure, e.g., in its prompts and in how the on-demand test measurement is acquired. In a CGM implementation, the CGM data may be acquired continuously or intermittently, and are typically available prior to the reference measurement.

A variation of the above procedure might be employed where an on-demand measurement is acquired prior to but recent to the reference measurement. In such a case, the system may check for this and not prompt the subject, and use the on-demand measurement that had already been acquired. Alternatively, the procedure may not use an explicit prompt, but the user could be instructed to perform the on-demand test measurement without the prompt. Furthermore, the receiver unit could provide option to include the prompt or not.

The on-demand test measurement may include one or more sensor measurements. These measurements may be temporal signal samples in the past, lagged measurements of the sensor signal such as can be achieved by measuring the same signal lagged by an RC circuit, or any other form of signal measurements including measurement of multiple signals. For example, sensor temperature may also be measured. As previously mentioned, specific embodiments for acquiring periodic, averaged and rate-of-change data from a transmitter device in the context of an on-demand measurement are discussed further below.

Some CGM calibration protocols use sensor data acquired prior to, substantially proximate to, and after the reference test reading in the sensitivity calculation. For example, in some embodiments, CGM data subsequent to a BG reading may be used to improve the lag correction included in the calibration method. Such data may be used to update the calibration at some time, for example about seven minutes, after the BG reading.

Still referring to FIG. 10, in certain embodiments, at a predetermined time after the reference measurement (1070), the receiver unit prompts the user to acquire another on-demand test measurement (1080). The receiver unit uses the newly acquired on-demand test measurement to generate an updated sensitivity factor (1090). This process may use the previously acquired on-demand data and reference measurement, or only the previous sensitivity results or other processing variations are possible as appropriate.

If the on-demand system has the capability of transmitting periodic, averaged or rate-of-change information based on a sequence of measurements preceding to the on-demand transmission, then that additional data will be available for use in connection with the above-described update, to further refine the update.

Certain embodiments of the present disclosure may include a method for calibrating a signal from an subcutaneously or transcutaneously positioned electrochemical sensor comprising generating a signal from the sensor, the signal corresponding to an analyte concentration level in a biofluid of a user, determining if the signal indicates a predetermined low or high analyte concentration level, prompting the user to assay a calibration sample of the user's blood to obtain a calibration value, if the signal indicates a high or low analyte concentration level, and relating the calibration value to at least one of the signals from the sensor.

In certain aspects, the high and low analyte concentration levels may be within a euglycemic range.

In certain aspects, the high analyte concentration level may be within an elevated analyte range.

In certain aspects, the analyte may be glucose, and further the elevated analyte range may be a hyperglycemic range.

In certain aspects, the high concentration level may be above 350 mg/dL.

In certain aspects, the low analyte concentration level may be within a depressed analyte range.

In certain aspects, the analyte may be glucose, and further the elevated analyte range may be a hypoglycemic range.

In certain aspects, the low concentration level may be below 60 mg/dL.

In certain aspects, the analyte may be glucose and both the low and high analyte concentration levels may be within a hyperglycemic range.

In certain aspects, the analyte may be glucose and both the low and high analyte concentration levels may be within a hypoglycemic range.

Certain embodiments may further include determining whether the prompted assay is within window of time for a prescheduled calibration prompt and skipping the prescheduled calibration prompt if the prompted assay is within window of time.

In certain aspects, the window of time may be three hours or less.

In certain aspects, the skipped prescheduled calibration prompt may be reset to occur at a time in the future

In certain aspects, the assayed calibration sample may be obtained from a fingerstick testing site.

In certain aspects, the assayed calibration sample may be obtained from an alternative site test.

Certain aspects may include storing the location of the testing site.

In certain aspects, the location may be located along a leg of a user.

In certain aspects, the location may be located along an abdomen of a user.

In certain aspects, obtaining the calibration measurement may comprise determining the calibration measurement in less than or equal to about 1 μL of blood.

In certain aspects, obtaining the calibration measurement may comprise determining the calibration measurement in less than or equal to about 0.5 μL of blood.

In certain aspects, obtaining the calibration measurement may comprise determining the calibration measurement in less than or equal to about 0.2 μL of blood.

In certain aspects, the predetermined low or high analyte concentration level may be calculated based upon a percentage of a user's average analyte level.

In certain aspects, the calibration value may be compared to at least one signal from the sensor for use in calibrating the sensor.

In certain aspects, the calibration value may be discarded if it is not within a predefined threshold of the at least one of the signals from the sensor.

In certain aspects, the calibration value may be weighted based upon the difference between the calibration value and the at least one signal from the sensor.

In certain aspects, the calibration value may be discarded if the absolute value of the rate of change of the current analyte value exceeds a threshold value.

In certain aspects, the subcutaneously or transcutaneously positioned electrochemical sensor may be a component of a continuous glucose monitoring system.

Certain embodiments of the present disclosure may include a method, comprising obtaining a reference data point at a first analyte concentration level, receiving a first data at the first analyte concentration level, calibrating the first data based on the reference data point, obtaining a second data at a second analyte level, updating the calibrated first data based on the second data, and calibrating the second data, wherein the first analyte concentration level if different from the second analyte concentration level.

Certain embodiments of the present disclosure may include an analyte monitoring device, comprising an operative component adapted to measure an analyte concentration from a sample obtained from a testing location of a user, and a receiver adapted to receive a signal from the operative component relative to the measured analyte concentration, wherein the receiver is configured to store information corresponding to the analyte concentration and the testing location to process analyte related signals based at least in part on the stored analyte concentration information and the testing location information.

In certain aspects, the receiver may include a user interface for providing the testing location information.

In certain aspects, the user interface may include one or more of a keyboard or a touch screen monitor to select the testing location from a database of testing locations.

In certain aspects, the touch screen monitor may display a physiological model to select the testing location from the physiological model, wherein the testing locations retrieved from the database is associated with the corresponding location displayed on the physiological model.

In certain aspects, one or more regions of the physiological model may be highlighted in response to manipulation of the user interface.

In certain aspects, the analyte may be glucose.

In certain aspects, the operative component may be an analyte test strip.

In certain aspects, the stored analyte level may be used to calibrate the analyte monitoring device.

In certain aspects, the testing location and corresponding analyte level concentration may be used determine or correct blood-to-interstitial glucose lag.

In certain aspects, the receiver may be a component of a continuous glucose monitoring system.

In certain aspects, the receiver may be configured to receive a signal from a transmitter in signal communication with an analyte sensor, where the received signal is indicative of an analyte level.

In certain aspects, the receiver may be a component of an on-demand glucose monitoring system.

In certain aspects, the testing location may be selected from the group comprising a hand, finger, palm, arm, abdomen, thigh, and a calf

In certain aspects, the receiver may be configured to output the testing location.

In certain aspects, the receiver may include a display to indicate the testing location.

In certain aspects, the display may include a physiological model that indicates the testing location.

In certain aspects, the display may include a textual message to indicate the testing location.

Certain embodiments of the present disclosure may include a method for calibrating an analyte monitor device, including measuring an analyte concentration from a testing location of a user, storing the analyte concentration and corresponding testing location information, and modifying a physiological model to correct for blood to interstitial glucose lag based on the testing location.

In certain aspects, the testing location may be one of a finger, an arm, leg, and abdomen.

In certain aspects, the testing location may be one of an upper arm, lower arm, calf, and thigh.

In certain aspects, a blood glucose test strip may measure the analyte concentration from a biological fluid of the user.

Certain aspects may include storing information corresponding to the analyte concentration and the testing location.

Certain aspects may include receiving user inputted testing location information and associating a corresponding analyte concentration level to the testing location information.

Certain embodiments of the present disclosure include a method for calibrating an analyte sensor, comprising retrieving a first calibration measurement, requesting a current calibration measurement, receiving the current calibration measurement, comparing the first calibration measurement to the current calibration measurement, and calibrating the analyte sensor based on one or more of the retrieved first calibration measurement or the received current calibration measurement if the current calibration measurement is outside a threshold value compared to the first calibration measurement.

In certain aspects, the threshold may include at least 50 mg/dL, at least 100 mg/dL, or greater than 150 mg/dL.

In certain aspects, the current calibration measurement may include a blood glucose measurement measured by a blood glucose monitor in response to the request for a current calibration measurement.

Certain aspects may include updating a calibration schedule if the current calibration measurement is outside a threshold value compared to the first calibration measurement.

In certain aspects, the calibration schedule may be only updated if the current calibration measurement is within a predetermined time period from a next scheduled calibration measurement.

In certain aspects, the predetermined time period may include 2 hours or less.

Certain aspects may include notifying a user if the current calibration measurement is not outside a threshold value compared to the first calibration measurement.

Certain aspects may include requesting a new calibration measurement if the current calibration measurement is outside a threshold value compared to the first calibration measurement.

Certain aspects may include waiting a predetermined time period prior to requesting a new calibration measurement.

In certain aspects, the predetermined time period may include at least 1 hour.

In certain aspects, the predetermined time period may include at least 2 hours.

Various other modifications and alterations in the structure and method of operation of this disclosure will be apparent to those skilled in the art without departing from the scope and spirit of the embodiments of the present disclosure. Although the present disclosure has been described in connection with particular embodiments, it should be understood that the present disclosure as claimed should not be unduly limited to such particular embodiments. It is intended that the following claims define the scope of the present disclosure and that structures and methods within the scope of these claims and their equivalents be covered thereby.

What is claimed is:

1. An analyte monitoring device, comprising:
an operative component adapted to measure an analyte concentration from a sample obtained from a testing location of a user; and
a receiver adapted to receive a signal from the operative component relative to the measured analyte concentration, wherein the receiver is configured to store information corresponding to the analyte concentration and the testing location to process analyte related signals based at least in part on the stored analyte concentration information and the testing location information.

2. The analyte monitoring device of claim 1, wherein the receiver includes a user interface for providing the testing location information.

3. The analyte monitoring device of claim 2, wherein the user interface includes one or more of a keyboard, or a touch screen monitor to select the testing location from a database of testing locations.

4. The analyte monitoring device of claim 3, wherein the touch screen monitor displays a physiological model to select the testing location from the physiological model, wherein the testing locations retrieved from the database is associated with the corresponding location displayed on the physiological model.

5. The analyte monitoring device of claim 4, wherein one or more regions of the physiological model are highlighted in response to manipulation of the user interface.

6. The analyte monitoring device of claim 1, wherein the analyte is glucose.

7. The analyte monitoring device of claim 1, wherein the operative component is an analyte test strip.

8. The analyte monitoring device of claim 1, wherein the stored analyte level is used to calibrate the analyte monitoring device.

9. The analyte monitoring device of claim 8, wherein the testing location and corresponding analyte level concentration is used determine or correct blood-to-interstitial glucose lag.

10. The analyte monitoring device of claim 1, wherein the receiver is a component of a continuous glucose monitoring system.

11. The analyte monitoring device of claim 10, wherein the receiver is configured to receive a signal from a transmitter in signal communication with an analyte sensor, where the received signal is indicative of an analyte level.

12. The analyte monitoring device of claim 1, wherein the receiver is a component of an on-demand glucose monitoring system.

13. The analyte monitoring device of claim 1, wherein the testing location is selected from the group comprising a hand, finger, palm, arm, abdomen, thigh, and a calf.

14. The analyte monitoring device of claim 1, wherein the receiver is configured to output the testing location.

15. The analyte monitoring device of claim 14, wherein the receiver includes a display to indicate the testing location.

16. The analyte monitoring device of claim 15, wherein the display includes a physiological model that indicates the testing location.

17. The analyte monitoring device of claim 15, wherein the display includes a textual message to indicate the testing location.

18. A method for calibrating an analyte sensor, comprising:
retrieving a first calibration measurement;
requesting a current calibration measurement;
receiving the current calibration measurement;
comparing the first calibration measurement to the current calibration measurement; and
calibrating the analyte sensor based on one or more of the retrieved first calibration measurement or the received current calibration measurement if the current calibration measurement is outside a threshold value compared to the first calibration measurement.

19. The method of claim 18, wherein the threshold includes one of at least about 50 mg/dL, at least 100 mg/dL, or greater than about 150 mg/dL.

22. The method of claim 18, wherein the current calibration measurement includes a blood glucose measurement measured by a blood glucose monitor in response to the request for a current calibration measurement.

23. The method of claim 18, further comprising updating a calibration schedule if the current calibration measurement is outside a threshold value compared to the first calibration measurement.

24. The method of claim 23, wherein the calibration schedule is only updated if the current calibration measurement is within a predetermined time period from a next scheduled calibration measurement.

25. The method of claim 24, wherein the predetermined time period includes 2 hours or less.

26. The method of claim 18, further comprising notifying a user if the current calibration measurement is not outside a threshold value compared to the first calibration measurement.

27. The method of claim 18, further comprising requesting a new calibration measurement if the current calibration measurement is outside a threshold value compared to the first calibration measurement.

28. The method of claim 27, further comprising waiting a predetermined time period prior to requesting a new calibration measurement.
